(19) United States
(12) Patent Application Publication
(10) Pub. No.: US 2023/0148901 A1
(43) Pub. Date: May 18, 2023

(54) SAMPLE COLLECTION DEVICE

(71) Applicants: A. Anand Ganesh, Pittsburgh, PA (US); James Sutcliff, Pittsburgh, PA (US); Jessica Helsten, Pittsburgh, PA (US); Kumar Rathinavelu, Pittsburgh, PA (US); StanwixForbes LLC, Pittsburgh, PA (US)

(72) Inventors: A. Anand Ganesh, Pittsburgh, PA (US); James Sutcliffe, Peterborough, ON (CA); Jessica Helsten, Atlanta, GA (US); Kumar Rathinavelu, Chennai, TN (IN)

(73) Assignee: StanwixForbes LLC, Pittsburgh, PA (US)

(21) Appl. No.: 17/619,628

(22) PCT Filed: Sep. 17, 2021
(86) PCT No.: PCT/US2021/050769

§ 371 Date: Dec. 16, 2021

Related U.S. Application Data
(60) Provisional application No. 63/133,442, filed on Jan. 4, 2021.
(60) Provisional application No. 63/080,767, filed on Sep. 20, 2020.

(51) Int. Cl. A61B 5/097; G01N 33/497

(52) U.S. Cl. CPC A61B 5/097; G01N 33/497

(57) ABSTRACT

A breath sampling tube has a pair of opposing holes at opposite ends and a bore extending therethrough to connect the pair of opposing holes with one another forming a passageway. A collector has a chamber therein and an opening for receiving one of the opposite ends of the breath sampling tube, so that the chamber is in fluid communication with the passageway. A sample collection solution is in in the chamber. The sample collection solution includes an indicator for indicating a quantity of breathed air that has been collected and a viral transport medium.

### CROSS REFERENCE TO RELATED APPLICATIONS

This application claims the benefit under 35 U.S.C. § 119(e) of co-pending U.S. Provisional Application No. 63/080,767 entitled “COLLECTION DEVICE FOR FIELD APPLICATION AND VIRAL CHARACTERIZATION” filed Sep. 20, 2020 and of co-pending U.S. Provisional Application No. 63/133,442 entitled “SAMPLE COLLECTION DEVICE” filed Jan. 4, 2021. Both applications are incorporated herein by reference.

### TECHNICAL FIELD

The subject disclosure is directed to systems, methods, and apparatus for collecting samples of infectious agents.

### BACKGROUND ART

Infectious respiratory diseases, such as SARS-CoV-2, cause millions of deaths globally and are commonly spread by droplets or aerosols. The disease, SARS-CoV-2, in particular, is the largest and most devastating pandemic of our time. The disease has a high rate of transmission, is associated with severe illness, and can result in a significant number of fatalities. As a new virus to the human population, it has a virtually limitless pool of susceptibilities with few established vaccines or treatments. As a result, the disease has created a severe threat to health care personnel, first responders, and general populations worldwide.

Testing for the SARS-CoV-2 virus was recognized from the beginning as a key strategy for tracking and controlling the pandemic and this remains true. Since the disease is readily and primarily transmitted by droplets and aerosols in exhaled breath and is most transmissible before individuals are aware of infection, there is a need for an increased and rapid development of effective means for detecting the levels of the virus that are in air and/or airborne droplets.

Given the overwhelming number of SARS-CoV-2 infected cases in the US and globally, there is an urgent need for disruptive diagnostic technologies able to safely capture, quantify, and analyze virus particles using a simple, affordable one-step process with high precision and accuracy.

Testing for SARS-CoV-2 was recognized from the beginning as a key strategy for tracking and controlling the pandemic and this remains true. The workhorse for testing has been polymerase chain reaction (PCR) analysis of subject samples for the presence of the SARS-CoV-2 ribonucleic acid (RNA) (i.e., for the virus's genetic material). By far the most common method for collecting samples for PCR analysis has been to use a long stick tipped with absorbent material (a swab) to probe deeply into the nasal passages or the oropharynx (oronasal sampling). This sampling method, while low cost and efficient in terms of patient throughput, suffers from several drawbacks.

The process of swabbing is uncomfortable and distressing for many people since the swab must be inserted deeply to contact sensitive mucosal or pharyngeal membrane. Anecdotally, the sampling procedure has been variously described as weird, painful, distressing, burning, and causing tearing, sneezing and headaches, etc. The deep probe can cause involuntary physical withdrawal or gag reflexes and, rarely injury.

In addition to issues of discomfort, oronasal swabbing does not always capture virus even though it can be present. Some studies estimate 25% false negatives from oronasal swab tests. This can be because the skill of the technician, because sampling missed more heavily infected areas nearby or, as is the case later in most infections, the foci of infection had moved out of the oronasal area into the lower respiratory tract.

Currently there are no proven methods to test the viral concentration in the flow of air. Rather, existing airflow testing procedures utilize surface area swabbing or, in some instances, charged plates that embedded in the airway. Unfortunately, the existing methods are limited because the collected viral particles on the plate is minimal. Accordingly, there is a need for an improved viral concentration detection or test device that replaces nasal swabbing.

### DISCLOSURE OF INVENTION

In various implementations, a sample collection apparatus includes a breath sampling tube having a pair of opposing holes at opposite ends and a bore extending therethrough to connect the pair of opposing holes with one another forming a passageway. A collector has a chamber therein and an opening for receiving one of the opposite ends of the breath sampling tube, so that the chamber is in fluid communication with the passageway. A sample collection solution is in in the chamber. The sample collection solution includes an indicator for indicating a quantity of breathed air that has been collected and a viral transport medium.

### BRIEF DESCRIPTION OF THE DRAWINGS

FIG. 1 is a perspective view of an exemplary embodiment of a sample collection device in relation to a user in accordance with this disclosure.

FIG. 2 is a cross-section view in side elevation of the exemplary embodiment shown in FIG. 1.

FIG. 3 is a side view of a vial that is part of the exemplary embodiment shown in FIG. 1.

FIG. 4 is a bottom view of the vial shown in FIG. 3.

FIG. 5 is a side view of a cap that is part of the exemplary embodiment shown in FIG. 1.

FIG. 6 is a top view of the cap that is shown in FIG. 5.

FIG. 7 is a side view of a connector that is part of the sample collection device shown in FIG. 1.

FIG. 8 is a bottom view of the connector shown in FIG. 7.

FIG. 9 is a side view of a base for a collector in accordance with the subject matter of the disclosure.

FIG. 10 is a bottom view of the vial shown in FIG. 9.

FIG. 11 is a side view of another embodiment of a base for a collector in accordance with the subject matter of the disclosure.

FIG. 12 is a bottom view of the vial shown in FIG. 11.

FIG. 13 is a cross section view in side elevation of a liquid collection kit in accordance with this disclosure.

FIG. 14 is an exemplary process in accordance with this disclosure.

FIG. 15 is an exemplary operation view with another embodiment.

FIG. 16 is a cross section view of the embodiment shown in FIG. 15.

FIG. 17 is a perspective view of the embodiment shown in FIG. 15.

FIG. 18 is a front view of the embodiment as shown in FIG. 17.

FIG. 19 is a top view of the embodiment as shown in FIG. 17.

FIG. 20 is a side view opposite of the view presented in FIG. 16.

FIG. 21 is a back view of the embodiment as shown in FIG. 17.

FIG. 22 is a bottom view of the embodiment as shown in FIG. 17.

### MODES FOR CARRYING OUT THE INVENTION

The disclosure is directed to a sample collection device that is particularly adapted to collect a sample that contains an infectious agent. In particular, the disclosure is directed to a sample collection device that receives a quantity of breathed air through a tube from an individual who can be infected with the infectious agent. The breathed air is placed in contact with a liquid that can hold the infectious agent therein and that can be tested for the presence of the infectious agent. The liquid includes an indicator that changes color based upon the quantity of breathed air. Once the sample is collected, the sample can be analyzed to determine the amount of infectious agent, such as viral particles, that are contained therein.

The detailed description provided below in connection with the appended drawings is intended as a description of examples and is not intended to represent the only forms in which the present examples can be constructed or utilized. The description sets forth functions of the examples and sequences of steps for constructing and operating the examples. However, the same or equivalent functions and sequences can be accomplished by different examples.

References to “one embodiment,” “an embodiment,” “an example embodiment,” “one implementation,” “an implementation,” “one example,” “an example” and the like, indicate that the described embodiment, implementation or example can include a particular feature, structure or characteristic, but every embodiment, implementation or example can not necessarily include the particular feature, structure or characteristic. Moreover, such phrases are not necessarily referring to the same embodiment, implementation or example. Further, when a particular feature, structure or characteristic is described in connection with an embodiment, implementation or example, it is to be appreciated that such feature, structure or characteristic can be implemented in connection with other embodiments, implementations or examples whether or not explicitly described.

Numerous specific details are set forth in order to provide a thorough understanding of one or more embodiments of the described subject matter. It is to be appreciated, however, that such embodiments can be practiced without these specific details.

Various features of the subject disclosure are now described in more detail with reference to the drawings, wherein like numerals generally refer to like or corresponding elements throughout. The drawings and detailed description are not intended to limit the claimed subject matter to the particular form described. Rather, the intention is to cover all modifications, equivalents and alternatives falling within the spirit and scope of the claimed subject matter.

The disclosure is directed to sample collection apparatuses, devices, systems, methods, and/or kits that are administered and accepted in a more reliable and easier manner. The disclosed breath-sampling instrumentalities replace and/or complement existing oronasal swabbing instrumentality.

The instrumentalities are configured to allow an individual to exhale through a straw-like tube that is immersed in a solution that is capable of carrying maintaining virus in a viable state. The solution is contained within a plastic sample vial, so that the solution can be transmitting for processing using standard PCR testing devices. The collection device can include a color-changing indicator that can be calibrated to the viral load that is contained within the solution.

In some implementations, a method of collecting a breath test sample is practiced. A quantity of breathed air is received from a person through a breath sampling tube having a passage extending therethrough. The quantity of breathed air is transported through the passage into a chamber defined by a collector with the chamber being in fluid communication with the passage. The quantity of breathed air is absorbed into a sample collection solution in the chamber with the sample collection solution including a pH indicator and a viral transport medium.

In other implementations, a kit for collecting samples includes a breath sampling tube having a pair of opposing holes at opposite ends and a bore extending therethrough to connect the pair of opposing holes with one another forming a passageway. A collector has a chamber therein and an opening for receiving one of the opposite ends of the breath sampling tube, so that the chamber will be in fluid communication with the passageway. A sample collection solution fills the chamber, at least partially. The sample collection solution includes a pH indicator and a viral transport medium.

While the disclosed collection instrumentalities are designed with a certain virus in mind, it is envisioned that such a device can be used in other applications where breath sampling is necessary. Modifications can be made to enable the device to detect COVID 19 and respiratory related viruses, perform alcohol breathalyzer test, and conduct pregnancy test using HcG Hormones.

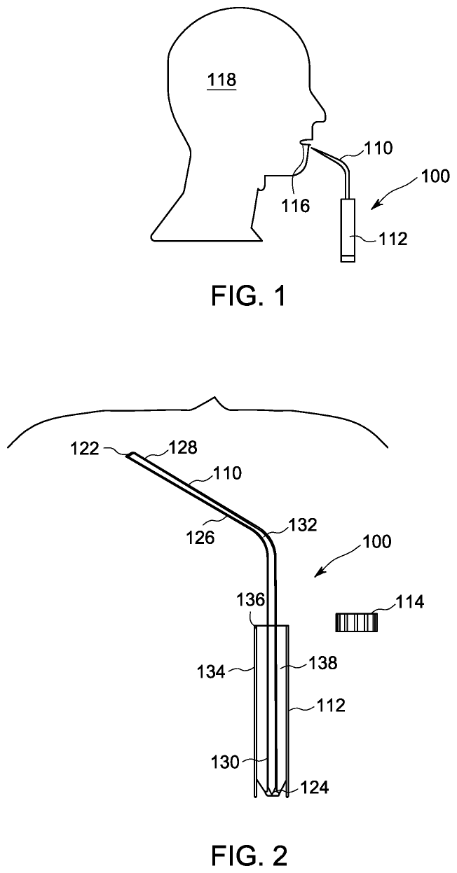

Referring to FIGS. 1-8, various features of the subject disclosure are now described in more detail with respect to a sample collection device, generally designated by the numeral 100, are shown. The device 100 includes an elongated breath sampling tube 110, a vial or tubular collector 112, and an optional cap 114. The device 100 is configured to receive a quantity of breathed air from the mouth 116 of a patient 118. In some embodiments, a connector 120 connects the breath sampling tube 110 to the collector 112.

The breath sampling tube 110 can be dimensioned in a manner to resemble a typical drinking straw, so that the patient 118 can produce a good seal with the mouth 116 when the patient 118 engages the breath sampling tube 110. In some embodiments, the tube length can be optimized to be a balance between ease of use and cost.

Referring to FIG. 2, the breath sampling tube 110 has a pair of opposing holes 122-124 and a bore 126 extending therethrough to connect the holes 122-124 to one another. The holes 122-124 are positioned at opposite ends 128-130 of the breath sampling tube 110, so that the holes 122-124 and the bore 126 form a passageway 132 the can transport fluid through the breath sampling tube 110. The end 128 inserts into the mouth 116 of the patient 118, so that air can be breathed through the hole 122 for transport through the passageway 132.

The end 130 inserts into the collector 112, so that the passageway 132 can transport the breathed air from the mouth 116 through the hole 124 into the collector 112. In some embodiments, the end 130 includes a plurality of micro-perforations and/or a cap (not shown) that includes a plurality of micro-perforations. In such embodiments, the micro-perforations that can have sizes ranging from about 1 μm to less than about 1 mm the cap can have a rough surface formed by sintered metal.

The collector 112 includes a body 134 and an opening 136 for receiving the breath sampling tube 110. The body 134 defines a chamber 138. The collector opening 136 is in fluid communication with the breath sampling tube hole 124, so that breathed air can be transported from the mouth 112, through the passageway 132, and into the chamber 138, so that the chamber 138 is in fluid communication with the mouth 112.

In some embodiments, an optional cap 114 is placed on the opening 136 after the collection process. The optional cap 114 provides additional sealing properties. In some embodiments, the optional cap 114 can allow the sampling tube 110 be inserted through during the collection process.

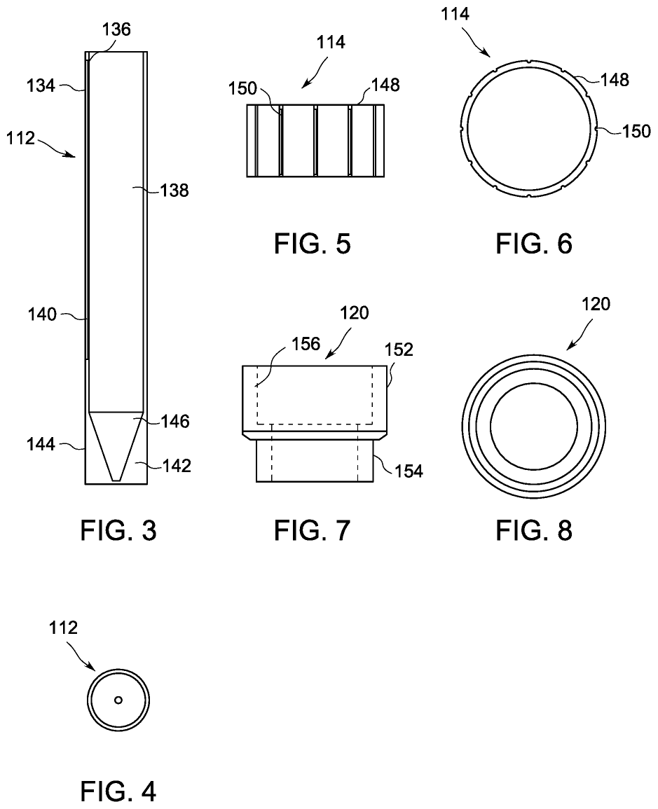

As shown in FIGS. 3-4, the collector 112 is an elongated tube having an extended portion 140 and a base 142. The base 142 includes an annular ring 144 that surrounds a conical extension 146. In some embodiments, the base 142 includes a sintered filter.

The chamber 138 holds a sample collection solution that provides the device 100 with the ability to collect infectious agent (i.e., virus) samples through liquid impingement. The sample collection solution includes an indicator, an antifoaming agent, and a viral transport medium. In this exemplary embodiment, the indicator is a pH indicator that indicates the quantity of breathed air that has been expelled from the mouth 116. The antifoaming agent can be desirable to prevent the viral transport medium from frothing when it flows through porous media with small holes therein. The sample collection solution can be prepared by mixing about 3.0 ml of viral transport medium with about 0.1 ml of indicator and about 0.15 mg antifoaming agent.

The indicator can be a pH indicator that includes a halochromic chemical compound that has the ability to visually indicate the pH of the sample collection solution. In some embodiments, the pH indicator can include bromothymol sulfone phthalein. In some embodiments, the indicator can be calibrated to produce color changes based upon a predetermined amount of virus in a sample based upon the fact that a standard quantity of breathed air for a particular breath averages about 500 ml of exhaled air. In such embodiments, the concentration of the indicator can be about 4%. The indicator can be provided by MilliporeSigma of St. Louis, Mo.

The viral transport medium can include fetal bovine serum (FBS), Hanks' Balanced Salt Solution (HBSS), antibiotics, antifungals, and phenol red. Such viral transport medium can include a saline solution that includes inorganic salts, glucose, and phosphate. An exemplary example of viral transport medium includes the viral transport medium sold by Rocky Mountain Biologicals, LLC of Missoula, Mont.

FBS is collected from unborn calves that are accidentally discovered after a pregnant cow has been processed. The blood has a low amount of immunoglobulin (antibodies) content and high concentration of essential components, such as hormones, transport proteins, and growth factor, for cell survival and proliferation. Bovine serum albumin is a major component that provides antioxidant, cryoprotectant, and anti-adsorption properties that favor retention of intact virus in solution over lysis and adherence to plastic. FBS can preserve viral host cells and to support viral preservation and amplification, ensuring quality samples for diagnostic testing. FBS can include either heat inactivated FBS or not inactivated FBS.

HBSS provides an isotonic solution to liquid media that contributes to the physiological requirements necessary for cell and viral stability. Variations of HBSS can include calcium, magnesium, and/or phenol red.

The antibiotics and the antifungals, such as gentamicin and amphotericin B, can keep liquid media free of contaminants. Bacteria and fungi from the respiratory tract and other sites can disrupt the viability of viral particles and/or degrade deoxyribonucleic acid (DNA) and RNA if allowed to proliferate.

The antifoaming agent can be any suitable antifoaming agent. In some embodiments, the antifoaming agent can be a foam suppressor for aqueous and non-aqueous systems. The antifoaming agent can include up to 100% active silicone polymer and, in some embodiments, does not include an emulsifier. The antifoaming agent can be provided as a concentrate that is typically effective at 1-100 ppm. An exemplary antifoaming agent includes Antifoam A Concentrate provided by MilliporeSigma of St. Louis, Mo. The antifoaming agent can be diluted to a final dilution of 50 ppm.

The viral transport medium is a non-hazardous mixture of buffered solutions and antimicrobials that preserves a virus and eliminates contaminant flora that might interfere with testing. The viral transport medium is compatible with a wide variety of clinical tests from PCR to direct antigen testing to culturing, allowing different tests to be run from the same sample.

In operation, the patient 118 breathes into the breath sampling tube, which carries the breath content into the collector 112. As the breath content contacts the sample collection fluid, which comprises of a viral transport medium, it can trigger a potential reaction from the indicator component within, if it contains the targeted virus. In some embodiments, the reaction is immediate and the patient 118 can see the results right away. In some other embodiments, the reaction requires some passage of time and the sample collection fluid can be collected in a lab for further analysis. The open distal end 136 of the collector 112 allows excess breath content to evacuate so as to avoid pressurizing the collector 112 in an enclosed space. In some embodiments, a check valve or filter component can be included on the distal end 136 of the collector.

As shown in FIGS. 5-6, the cap 114 is an essentially annular ring 148 having a plurality of ridges 150. The cap 114 can encircle the breath sampling tube 110 to close the tubular collector 112 to seal the sample collection solution therein. In some embodiments, the breath sampling tube 110 includes a check valve or a one-way valve to prevent the patient 118 from drawing the sample collection solution into the mouth 116.

As shown in FIGS. 7-8, the connector 120 is a tubular annular ring. The connector 120 includes an upper tubular portion 152, a lower tubular portion 154, and a bore 156 extending therethrough. The upper portion 152 has a greater outer diameter and a greater inner diameter than the lower tubular portion 154. The connector 120 can be inserted into the tubular collector 112 and receive the breath sampling tube 110.

Components of the device 100 can be made from any suitable material through any suitable manufacturing method. Suitable materials include flexible, rigid, or semi-rigid materials. Suitable materials also include metals, ceramics, plastics, composites, and/or combinations thereof. In some embodiments, the tubular collector 112 can be made from transparent, semi-transparent, and/or translucent materials. In other embodiments, the breath sampling tube 110 can be made from metal. In such embodiments, the cap 114 can be made from metal or plastic.

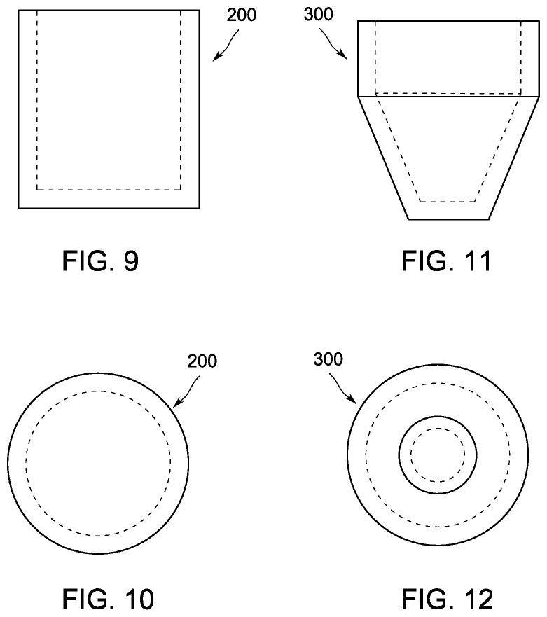

Referring now to FIGS. 9-12 with continuing reference to the foregoing figures, additional embodiments of a base, generally designated by the numerals 200 and 300, are shown. Like the embodiment shown in FIGS. 1-8, the bases 200 and 300 are part of a collector, such as the collector 112. The bases 200 and 300 can be connected to an extended portions, such as the extended portion 140 shown in FIGS. 1-8.

The connection can be releaseable or permanent, such as when the bases 200 and 300 are integral, unitary, or otherwise formed with the extended portion. Unlike the embodiments shown in FIGS. 1-8, the base 200 is essentially cylindrical. The base 300 is essentially frustoconical. It is understood that a person skilled in the art could have variations of the base for this section of the collector 112. It can be inferred that other geometrical variations can be applied to this section, depending on the particular need of the application.

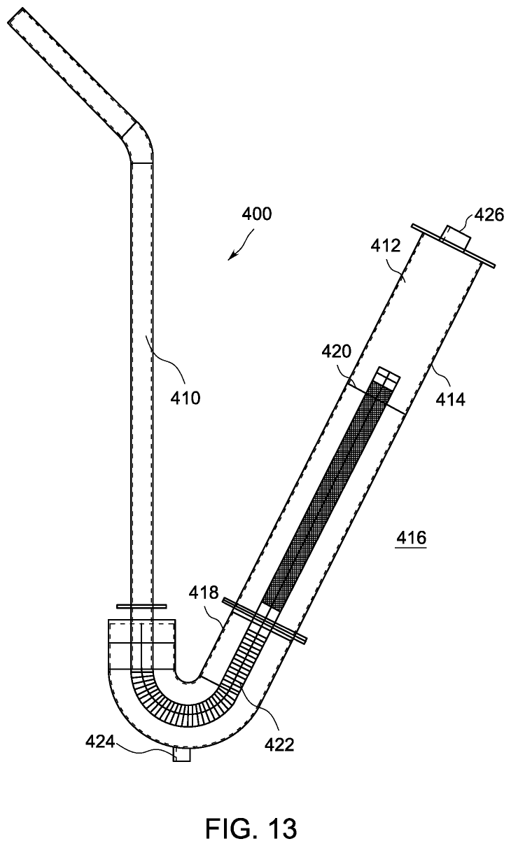

Referring now to FIG. 13 with continuing reference to the foregoing figures, a collection kit, generally designated by the numeral 400, is shown. The collection kit 400 is configured to receive a quantity of breathed air from a mouth, such as the mouth 116 of the patient 118, shown in FIG. 1. In some embodiments, the collection kit 400 can be configured to perform testing procedures can be used to test the viral concentration of airflow accurately. The collection kit 400 can be used with any source, such as a ventilator (not shown) or airflow output from a disinfecting device (not shown).

The collection kit 400 includes an inlet tube 410 and a tubular collector 412. The collector 412 an elongated essentially cylindrical outer shell 414 having an elongated essentially cylindrical sample container 416 positioned therein. A flexible portion 418 connecting the cylindrical outer shell 414 to the inlet tube 410. A spacer 420 holds the sample container 416 within the cylindrical outer shell 414. A flexible tube 422 connects the sample container 416 to the inlet tube 410, so that the inlet tube 410 is in fluid communication with the sample container 416. The sample container 416 can hold sample collection solution therein.

The collector 412 includes a pair of spouts 424-426. The spout 424 is positioned on the flexible portion 418 to drain the collector 412. The spout 426 is configured to connect to reservoir (not shown).

In this embodiment, the sample container 416 can contain sample collection fluid, as described previously in reference to FIG. 1-12. This allows ease of transporting the sample collected in a lab setting, such that at least one sample container 416 can be swapped and used in connection with the flexible portion 418. In some embodiments, the tubular collector 412 can be easily removed from the flexible portion 418 to enable convenient exchange of multiple sample containers 416.

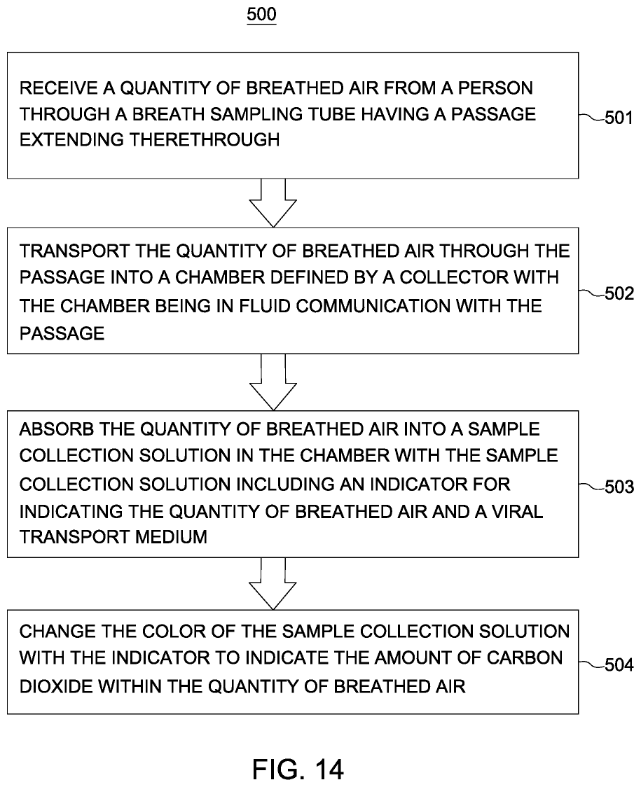

Referring now to FIG. 14 with continuing reference to the foregoing figures, an exemplary method, generally designated with the numeral 500, for collecting a sample that can contain an infectious agent is shown. The method 500 can be performed using the sample collection device 100 shown in FIGS. 1-8, the sample collection device configured with a collector having one of the bases 200 and 300 shown in FIGS. 9-12, and/or the sample collection kit 400 shown in FIG. 13.

At 501, a quantity of breathed air from a person is received through a breath sampling tube having a passageway extending therethrough. In this exemplary embodiment, the breath sampling tube can be the breath sampling tube 110 shown in FIGS. 1-8. The passageway can be the passageway 132 shown in FIGS. 1-8.

At 502, the quantity of breathed air is transported through the passage into a chamber defined by a collector with the chamber being in fluid communication with the passage. In this exemplary embodiment, the collector can be the collector 112 shown in FIGS. 1-8.

At 503, the quantity of breathed air is absorbed into a sample collection solution in the chamber with the sample collection solution including an indicator for indicating the quantity of breathed air and a viral transport medium. In this exemplary embodiment, the chamber can be the chamber 138 shown in FIGS. 1-8.

At 504, the color of the sample collection solution is changed with the indicator to indicate the amount of carbon dioxide within the quantity of breathed air.

The method for collecting and testing samples can be applied to a variety of configurations of a collection device. In the embodiment described in FIG. 1-12, the sample collection solution resides within the same chamber as the breath sampling tube. In the embodiments described in FIG. 13, the sample collection solution resides in a sample container in a separate sample container. It is understood that further variation could be constructed to facilitate the process described herein.

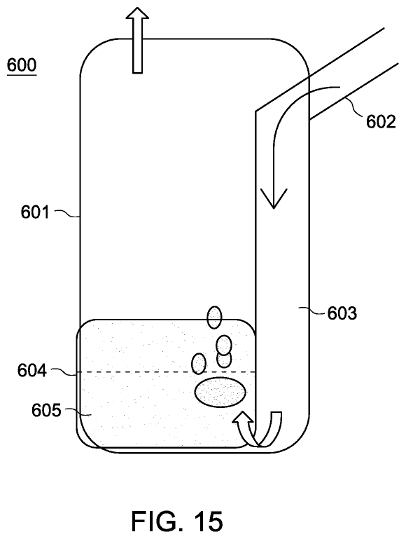

Referring to FIG. 15, an exemplary embodiment of the sample collection kit is shown with continuing reference to the foregoing figures. The figure illustrates the function of a cartridge sample collection kit 600, which operates based on the same method and principle demonstrated in collection device 100 and collection kit 400. In this embodiment, the cartridge sample collection kit 600 is configured to include key components from previous embodiments in a compact fashion.

The sample collection kit 600 includes a collection container 601, which is contains sample collection fluid 605. A breath sampling tube 602 is located on the upper side position of the collection container 601, and extends into its interior. An internal baffle 603 connects to the breath sampling tube 602 on one side of its perimeter, effectively extending the breath sampling tube into the collection container 601. The internal baffle 603 divides the collection container 601 essentially into two sections on its interior.

In one embodiment, the internal baffle 603 does not connect with bottom of the collection container 601. Thus, a pathway is open between two sections within the collection container 601, allowing viral transport medium 605 to reach both sections. In some embodiment, a mesh or screen could be installed to extend the baffle 603 to the bottom of the collection container 601 while still allowing access between two sections.

A filtering structure 604 is on the other side of the internal baffle, opposite of space directly connected to the breath sampling tube 602. The filtering structure 604 is comprised of an array of small sticks, in one embodiment. In other embodiments, varied width and size of the sticks can be used to provide specific filtering needs.

In operation, a user 118 as depicted in FIG. 1, would make contact with the breath sampling tube 602, and breath into said sampling tube. With continuing reference to ongoing figures and previous embodiments, a user would supply breathing sample into the collection container 601. Airflow would allow breath sample to travel along the internal baffle 603 into the viral transport medium fluid 605 stored therein.

As the user 118 breaths into the sample collection kit 600, the viral transport fluid 605 would produce air bubbles with the introduction of air within. As the air bubbles make contact with the filter structure 604, the bubbles will break into smaller ones. This creates a plurality of smaller bubbles that would result in larger surface area in total, which improves the desolation rate of the process. In one embodiments, an outlet port would allow excessive volume of air evacuate through the sample collection kit 600.

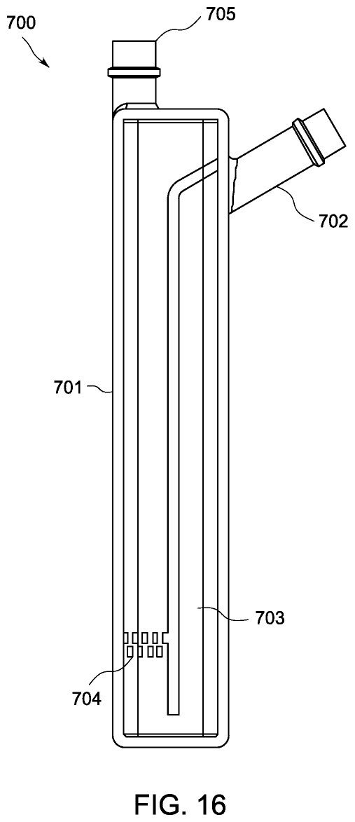

Referring to FIG. 16 for an exemplary embodiment of the sample collection kit as described in FIG. 15. This embodiment is a design based on the concept in FIG. 15, and a person skilled in the art would be able to modify certain aspects according to particular application needs. It is a cross section view showing the internal structures.

This exemplary embodiment is generally designated as cartridge sample collection device 700. As shown in FIG. 16, it includes a breath collection tube 702, which extends from the exterior of the device. The breath collection tube 702 acts as an inlet to the collection device 700, allowing a user to input breath sample into the interior of the device. The internal baffle 703 is positioned along the breath collection tube 702, ensuring a pathway into interior of the device 700 from opening of breath collection tube 702. On opposite side of the internal baffle 703, a filtering structure 704 is installed between it and the enclosure 701 of the device 700. As described in FIG. 15, variations of arrangements can be made to form the filtering structure 704 based on application needs. In operation, a sample collection fluid would be stored within the enclosure 701 to facilitate particle collection. In one embodiment, this could be viral transport medium fluid 605 as described in FIG. 15. An outlet 705 is located on top of the device 700, such that excessive volume of air can be evacuated in use.

In some embodiments, a check valve can be installed in connection with the outlet, to ensure a sufficient level of containment of the breath content with the collection kit. This would allow excessive breath content to escape without compromising the sample containment function of the collection kit.

In some embodiments, a check valve can be installed in connection with the breath sampling tube, such that the flow path can be limited to one direction. This provides heightened safety for the user, by preventing backwards airflow after the breath sample has been provided.

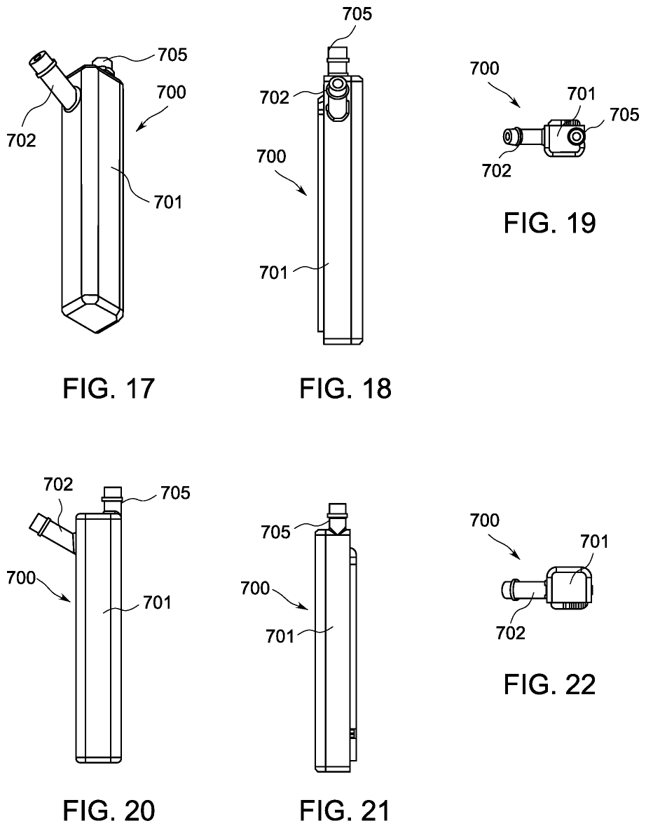

Referring to FIG. 17-22, various views of the sample collection device 700 are presented. In this embodiment, the design is created to achieve a level of compact ergonomic geometry that promotes a certain level of manufacturing and marketing need. It is envisioned that this design would enable a user to collect breath test sample in the process outlined herein with convenience. In some embodiments, check valves or flow regulating means could be installed along the inlet and outlet section of the device.

The embodiment illustrated in FIG. 16-22 can be understood as a compact version of the collector 100 and 400. The general concept and operation method follows the flow chart outlined in FIG. 15, wherein a breath sample is taken from an input and directed into the collector that contains a sample collection fluid, and the outlet evacuates any excessive quantity the breath content. Whereas collection kit 100 has a breath sampling tube separate from the collector chamber, and the collection kit 400 has a collector that is detachable from the breath sampling tube, the collection kit 700 internalizes the flow path and forms a compact structure based on the same operating principle. It can be understood that the method illustrated in FIG. 15 can be further applied in other embodiments according to specific user requirements.

The detailed description provided above in connection with the appended drawings explicitly describes and supports various features of a sample collection device. By way of illustration and not limitation, supported embodiments include a sample collection apparatus comprising: a breath sampling tube having a pair of opposing holes at opposite ends and a bore extending therethrough to connect the pair of opposing holes with one another forming a passageway, a collector having a chamber therein and an opening for receiving one of the opposite ends of the breath sampling tube, so that the chamber is in fluid communication with the passageway, and a sample collection solution in the chamber, wherein the sample collection solution includes an indicator for indicating a quantity of breath that has been collected and a viral transport medium.

Supported embodiments include the foregoing sample collection apparatus, wherein the collector is an elongated tubular collection vial.

Supported embodiments include any of the foregoing sample collection apparatuses, further comprising: a connector for connecting the breath sampling tube to the collector.

Supported embodiments include any of the foregoing sample collection apparatuses, wherein the breath sampling tube includes a one way valve for restricting fluid flow in one direction through the passageway.

Supported embodiments include any of the foregoing sample collection apparatuses, wherein the collector has an essentially cylindrical body.

Supported embodiments include any of the foregoing sample collection apparatuses, wherein the collector includes a base having an outer configuration selected from the group consisting of an essentially cylindrical shape, a conical shape, and a frustoconical shape.

Supported embodiments include any of the foregoing sample collection apparatuses, wherein the pH indicator includes a halochromic chemical compound.

Supported embodiments include any of the foregoing sample collection apparatuses, wherein the indicator is a pH indicator that has the ability to visually indicate the pH of the sample collection solution.

Supported embodiments include any of the foregoing sample collection apparatuses, wherein the pH indicator includes bromothymol sulfone phthalein.

Supported embodiments include any of the foregoing sample collection apparatuses, wherein the sample collection solution includes an antifoaming agent.

Supported embodiments include any of the foregoing sample collection apparatuses, wherein the antifoaming agent includes an active silicone polymer.

Supported embodiments include any of the foregoing sample collection apparatuses, wherein the viral transport medium is a saline solution.

Supported embodiments include any of the foregoing sample collection apparatuses, wherein the viral transport medium includes inorganic salts, glucose, and phosphate.

Supported embodiments include any of the foregoing sample collection apparatuses, wherein the one of the opposite ends of the breath sampling tube that inserts into the collector opening includes a plurality of micro-perforations.

Supported embodiments include any of the foregoing sample collection apparatuses, wherein the one of the opposite ends of the breath sampling tube that inserts into the collector opening includes an end cap having a plurality of micro-perforations therein.

Supported embodiments include any of the foregoing sample collection apparatuses, wherein the one of the opposite ends of the breath sampling tube that inserts into the collector opening includes an end cap having a sintered filter therein.

Supported embodiments include a system, a method, a kit, and/or means for implementing any of the foregoing sample collection apparatuses or a portion thereof.

Supported embodiments include a method of collecting a breath test sample, the method comprising: receiving a quantity of breathed air from a person through a breath sampling tube having a passage extending therethrough, transporting the quantity of breathed air through the passage into a chamber defined by a collector with the chamber being in fluid communication with the passage, and absorbing the quantity of breathed air into a sample collection solution in the chamber with the sample collection solution including an indicator for indicating the quantity of breathed air and a viral transport medium.

Supported embodiments include the foregoing method, further comprising: changing the color of the sample collection solution with the indicator to indicate the quantity of breathed air.

Supported embodiments include any of the foregoing methods, further comprising: changing the color of the sample collection solution with the indicator to indicate the amount of carbon dioxide within the quantity of breathed air.

Supported embodiments include an apparatus, a system, a kit, and/or means for implementing any of the foregoing methods or a portion thereof.

Supported embodiments include a kit for collecting samples comprising: a breath sampling tube having a pair of opposing holes at opposite ends and a bore extending therethrough to connect the pair of opposing holes with one another forming a passageway, a collector having a chamber therein and an opening for receiving one of the opposite ends of the breath sampling tube, so that the chamber will be in fluid communication with the passageway, and a sample collection solution for filling the chamber, at least partially, wherein the sample collection solution includes an indicator that changes color based upon a quantity of air that is breathed through the breath sampling tube and a viral transport medium.

Supported embodiments can provide various attendant and/or technical advantages in terms of a sample collection device that requires less technical know-how and infrastructure to use than oronasal sampling and, if appropriate, can be done by the subject itself.

Supported embodiments include a sample collection device that is far less uncomfortable to use than oronasal sampling and has none of the risk of injury or negative after-effects with which oronasal sampling is sometimes associated.

Supported embodiments include a sample collection device that is less intimidating and more acceptable to those considering being tested.

Supported embodiments include a sample collection device that is more attractive to health care and frontline works who must be testing regularly and whose alternatives, depending on the situation, are frequent oronasal sampling or one of the rapid tests that are known to have high false negative rates.

Supported embodiments include a sample collection device that incorporates tested and true PCR analysis of the sample thus reducing the probability of false negatives or positives.

Supported embodiments include a sample collection device that can acquire a whole breath sample that includes virus from all parts of the respiratory tract (not just the nasopharynx). Such devices are more effective than oronasal sampling for detecting older infections in which infectious load has moved into the lungs and bronchi.

The detailed description provided above in connection with the appended drawings is intended as a description of examples and is not intended to represent the only forms in which the present examples can be constructed or utilized.

It is to be understood that the configurations and/or approaches described herein are exemplary in nature, and that the described embodiments, implementations and/or examples are not to be considered in a limiting sense, because numerous variations are possible.

The specific processes or methods described herein can represent one or more of any number of processing strategies. As such, various operations illustrated and/or described can be performed in the sequence illustrated and/or described, in other sequences, in parallel, or omitted. Likewise, the order of the above-described processes can be changed.

Although the subject matter has been described in language specific to structural features and/or methodological acts, it is to be understood that the subject matter defined in the appended claims is not necessarily limited to the specific features or acts described above. Rather, the specific features and acts described above are presented as example forms of implementing the claims.

1-24. (canceled)

25. A sample collection solution for providing a sample collection device with the ability to collect an infectious agent sample from a patient through liquid impingement, the sample collection solution comprising:
a viral transport medium;
an indicator for indicating the quantity of breathed air that has been expelled from a mouth of a the patient; and
an antifoaming agent for preventing the sample collection solution from frothing when it flows through porous media with small holes therein.

26. The sample collection solution of claim 25, wherein the indicator is a pH indicator.

27. The sample collection solution of claim 26, wherein the pH indicator includes a halochromic chemical compound that has the ability to visually indicate the pH of the sample collection solution.

28. The sample collection solution of claim 27, wherein the pH indicator includes bromothymol sulfone phthalein.

29. The sample collection solution of claim 25, wherein the indicator is calibrated to produce color changes based upon a predetermined amount of virus in a predetermined sample collection solution sample size.

30. The sample collection solution of claim 25, wherein the antifoaming agent is a foam suppressor.

31. The sample collection solution of claim 30, wherein the antifoaming agent is a foam suppressor for a system selected from the group consisting of aqueous systems and non-aqueous systems.

32. The sample collection solution of claim 25, wherein the antifoaming agent includes silicone polymer.

33. The sample collection solution of claim 25, wherein the antifoaming agent includes an emulsifier.

34. The sample collection solution of claim 25, wherein the antifoaming agent can suppress foams when the concentration of the antifoaming agent is between 1-100 parts per million.

35. The sample collection solution of claim 25, wherein the antifoaming agent is diluted to a dilution of 50 parts per million.

36. The sample collection solution of claim 25, wherein the viral transport medium include at least one buffered solution and at least one antimicrobial.

37. The sample collection solution of claim 25, wherein the viral transport medium is a saline solution.

38. The sample collection solution of claim 37, wherein the viral transport medium includes inorganic salts, glucose, and phosphate.

39. In a sample collection device having a collector with a chamber for holding sample collection solution therein, the sample collection solution comprising:
a viral transport medium;
an indicator for indicating the quantity of breathed air that has been expelled from a mouth of a the patient into the sample collection device collector chamber; and
an antifoaming agent for preventing the sample collecti6on solution from frothing when it flows through porous media with small holes therein;
wherein the porous media is in the sample collection device.

40. The sample collection solution of claim 39, wherein the indicator is a pH indicator.

41. The sample collection solution of claim 40, wherein the antifoaming agent is a foam suppressor.

42. The sample collection solution of claim 41, wherein the viral transport medium include at least one buffered solution and at least one antimicrobial.

43. The sample collection solution of claim 42, wherein the viral transport medium is a saline solution.

44. The sample collection solution of claim 43, wherein the viral transport medium includes inorganic salts, glucose, and phosphate.
